(19) United States
(12) Patent Application Publication
(10) Pub. No.: US 2022/0288201 A1
(43) Pub. Date: Sep. 15, 2022

(54) COMBINATION THERAPY FOR TREATING RAS-MUTANT CANCERS

(71) Applicant: H. Lee Moffitt Cancer and Research Institute, Inc., Tampa, FL (US)

(72) Inventor: Kenneth Yee TSAI, Tampa, FL (US)

(21) Appl. No.: 17/628,920

(22) PCT Filed: Jul. 21, 2020
(86) PCT No.: PCT/US2020/042856

§ 371 Date: Jan. 21, 2022

Related U.S. Application Data
(60) Provisional application No. 62/877,010, filed on Jul. 22, 2019.

(51) Int. Cl. A61K 39/395; A61K 31/437; A61K 31/506; A61P 35/00

(52) U.S. Cl. CPC A61K 39/3955; A61K 31/437; A61K 31/506; A61P 35/00

(57) ABSTRACT

RAS mutation driven tumors are difficult to treat, with no targeted agents advancing to late stage clinical trials. Although immunotherapy is indicated for RAS mutant tumors, methods are needed to sensitize tumors to immunotherapy or to treat resistance to immunotherapy. Disclosed herein is a method for treating a RAS mutant cancer in a subject that involves administering to the subject a therapeutically effective amount of a BRAF inhibitor (BRAFi) in combination with immunotherapy, such as a checkpoint inhibitor. Also disclosed herein is a composition comprising a BRAFi and a checkpoint inhibitor in a pharmaceutically acceptable carrier. As disclosed herein, selective BRAF inhibition paradoxically prevents the growth of RAS mutant tumor cell lines in vitro with a consistent ERK hyperactivation and increased senescence.

### CROSS-REFERENCE TO RELATED APPLICATIONS

This application claims benefit of International Patent Application PCT/US2020/042856 filed Jul. 21, 202, which claims priority to U.S. Provisional Application No. 62/877,010, filed Jul. 22, 2019, both of which are hereby incorporated herein by reference in its entirety.

### BACKGROUND

The advent of targeted therapy based upon molecular dissection of tumor dependencies has revolutionized cancer therapy. This is nowhere more evident than for melanoma for which BRAF/MEK inhibition is extremely effective (Roskoski, R., Jr. Pharmacol Res 135:239-258 (2018)). However, NRAS-mutant melanomas, remain intractable in terms of targeted therapy. More generally, RAS-mutant tumors have been very difficult to treat, with no targeted agents advancing to late stage clinical trials. While immune-checkpoint blockade (ICB) is indicated for NRAS-mutant melanomas (Sarkisian, S. & Davar, D. Drug Des Devel Ther 12:2553-2565 (2018)), methods are needed to sensitize tumors to ICB or to treat initial or acquired resistance to ICB.

### SUMMARY

Although there are several genomically-defined subsets of melanoma, the molecular underpinnings of these diverse sets converge, at least in part, on ERK pathway signaling. Initial therapeutic responses coincide with massive decreases in ERK pathway signaling and resistance is often signaled by reactivation of ERK signaling (Paraiso, K. H. et al. Br J Cancer 102:1724-30 (2010); Paraiso, K. H. et al. Cancer Res (2011); Flaherty, K. T. et al. N Engl J Med 363:809-19 (2010)). Interestingly, it was also observed early on that in BRAF-wild type cells, BRAFi can activate the pathway instead (Hatzivassiliou, G. et al. Nature 464:431-5 (2010); Poulikakos, P. I., et al. Nature 464:427-30 (2010); Heidorn, S. J. et al. Cell 140:209-21 (2010); Halaban, R. et al. Pigment Cell Melanoma Res 23:190-200 (2010); McKay, M. M., et al. Curr Biol 21:563-8 (2011); Adelmann, C. H. et al. Oncotarget 7:30453-60 (2016); Karreth, F. A., et al. Mol Cell 36:477-86 (2009)). In fact, this phenomenon is likely to reflect general intrinsic biochemical properties of kinase inhibitors. This “paradoxical” ERK activation has been attributed to BRAFi-induced stabilization of RAS/RAF signaling complexes and resulting activation of ERK signaling in BRAF wild-type contexts (Hatzivassiliou, G. et al. Nature 464:431-5 (2010); Poulikakos, P. I., et al. Nature 464:427-30 (2010); Heidorn, S. J. et al. Cell 140:209-21 (2010); Halaban, R. et al. Pigment Cell Melanoma Res 23:190-200 (2010)). Additionally, it is reported that this activation requires intact CRAF activity (Hatzivassiliou, G. et al. Nature 464:431-5 (2010); Poulikakos, P. I., et al. Nature 464:427-30 (2010); Heidorn, S. J. et al. Cell 140:209-21 (2010); Halaban, R. et al. Pigment Cell Melanoma Res 23:190-200 (2010); Degirmenci, U., et al. Cells 9 (2020); Vin, H. et al. Elife 2, e00969 (2013)). According, the paradoxical activation of ERK fails to occur following the exposure of cells to so-called pan-RAF inhibitors, which simultaneously suppress BRAF and CRAF activity. Indeed, a spate of such inhibitors have been specifically developed over the last several years in an attempt to treat RAS-mutant cancer as single agents, but as of 2020, none has been approved for clinical use in humans (Degirmenci, U., et al. Cells 9 (2020)).

The phenomenon of paradoxical ERK activation accounts for some of the systemic toxicities associated with BRAFi treatment, most notably the induction of cutaneous squamous cell carcinoma (cuSCC), which occurs in 22% of patients treated with the BRAFi vemurafenib and 6% in patients treated with the BRAFi dabrafenib, averaged across multiple studies (Vin, H. et al. Elife 2, e00969 (2013); Menzies, A. M., et al. Pigment Cell Melanoma Res 26:611-5 (2013)). Intriguingly, RAS mutations are enriched in BRAFi treatment associated cuSCC. Approximately 60% of cuSCC from vemurafenib treated patients harbor activating RAS mutations compared 10-20% in sporadic cuSCC tumors (Oberholzer, P. A. et al. J Clin Oncol 30:316-21 (2012); Pickering, C. R. et al. Clin Cancer Res 20:6582-92 (2014); South, A. P. et al. J Invest Dermatol 134:2630-8 (2014); Su, F. et al. N Engl J Med 366:207-15 (2012); Chitsazzadeh, V. et al. Nat Commun 7:12601 (2016); Li, Y. Y. et al. Clin Cancer Res 21:447-56 (2015)). This enrichment of RAS mutations purportedly occurs because the presence of active RAS sensitizes RAS/RAF signaling modules to the ERK activating effects of BRAFi treatment (Hatzivassiliou, G. et al. Nature 464:431-5 (2010); Poulikakos, P. I., et al. Nature 464:427-30 (2010); Heidorn, S. J. et al. Cell 140:209-21 (2010); Halaban, R. et al. Pigment Cell Melanoma Res 23:190-200 (2010))). Other cancers with activating alterations in RAS, including leukemia and colorectal carcinoma, have also been reported in patients treated with BRAFi (Andrews, M. C. et al. J Clin Oncol 31:e448-51 (2013); Callahan, M. K. et al. N Engl J Med 367:2316-21 (2012)). Based on these findings, BRAFi are associated with the induction or acceleration of RAS mutant tumor growth and have therefore been considered contraindicated for the treatment of BRAF wild-type tumors. Consonant with that concept, BRAF-selective inhibitors, whether in combination with MEK inhibitors or not, are specifically indicated only for the treatment of BRAF-mutant cancers and not RAS-mutant cancers.

While oncogene-induced senescence due to massive overexpression of mutant RAS has been described as an important tumor suppression mechanism in its canonical form, the concept of senescence more generally has evolved over time to include additional variants of senescence including therapy-induced senescence (Ito, Y., et al. Trends Cell Biol 27:820-832 (2017); Lasry, A. & Ben-Neriah, Y. Trends Immunol 36:217-28 (2015); Ewald, J. A., et al. J Natl Cancer Inst 102:1536-46 (2010); Mooi, W. J. & Peeper, D. S. N Engl J Med 355:1037-46 (2006)). With this in mind and taking these sets of observations, the question was asked whether paradoxical ERK activation, as engendered by BRAFi exposure in the context of RAS-mutant cancers, could cause elevation of ERK pathway signaling to the point of causing senescence-like growth arrest akin to oncogene-induced senescence. Such activity specifically requires intact CRAF activity, which is in turn required for the paradoxical ERK activation. As disclosed herein, BRAFi treatment suppresses the growth of RAS-mutant melanoma by elevating ERK activity.

Therefore, disclosed herein is a method for treating a RAS mutant cancer in a subject that involves administering to the subject a therapeutically effective amount of a selective BRAF inhibitor (BRAFi) in combination with immunotherapy. In some embodiments, the BRAFi is administered in an amount effective to activate ERK.

Also disclosed herein is a method for treating a RAS mutant cancer in a subject that involves administering to the subject a therapeutically effective amount of an ERK activator in combination with immunotherapy.

In some embodiments, the immunotherapy is administered at least 1, 3, 4, 5, 6, 6, 7, 8, 9, 10, 11, 12, 13, 14, 15, 16, 17, 18, 19, 20, 21, 22, 23, 24 hours before the BRAFi. In some embodiments, the BRAFi is administered at least 1, 3, 4, 5, 6, 6, 7, 8, 9, 10, 11, 12, 13, 14, 15, 16, 17, 18, 19, 20, 21, 22, 23, 24 before the immunotherapy. In some embodiments, the BRAFi and immunotherapy are administered simultaneously. For example, in some embodiments, the BRAFi and immunotherapy are in the same composition. Therefore, also disclosed herein is a composition comprising a BRAFi and a checkpoint inhibitor in a pharmaceutically acceptable carrier.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. BRAF inhibitor treatment blocks the growth of RAS-mutant melanoma cell lines at sub-micromolar concentrations. (A-D) Colony growth assay IC50 measurements from eight melanoma cell lines treated with four BRAF inhibitors. A375 (gray) is a highly sensitive BRAF mutant melanoma cell line included as a positive control. All other lines are BRAF wild-type and have activating mutations in RAS. Lighter colored bars represent BRAF inhibitor ‘sensitive’ lines.

FIG. 2. BRAF inhibitor treatment activates ERK and blocks proliferation in RAS-mutant melanoma. (A) Phospho- and total MEK and ERK levels in 501mel and meIJUSO lysates after 72 hour treatment with DMSO (‘Ø’), vemurafenib (‘Ve,’ 1 μM), dabrafenib (‘Da,’ 200 nM), encorafenib (‘En,’ 200 nM), or PLX8394 (‘Px,’ 500 mM). (B) Apoptosis measured by PARP western blot after 72 hr treatment with dabrafenib (‘Da,’ 200 nM), encorafenib (‘En,’ 200 nM), or trametinib (‘Tm,’ 500 nM), a MEK inhibitor and positive control. The presence of a cleaved PARP band is a marker of apoptosis. (C-D) Representative images and quantitation of EdU proliferative staining with cells treated 72 hr with DMSO, dabrafenib (‘Dab,’ 200 nM), or encorafenib (‘Enco,’ 200 nM). Quantitation was with Nexelcom Celigo. (E-F) Representative images and quantitation of EdU proliferative staining with cells treated 1 week with DMSO (‘Ø’), dabrafenib (‘Dab,’ 200 nM), or encorafenib (‘Enco,’ 200 nM). Quantitation was with ImageJ (NIH). (G) ERK signaling and p21 expression in lysates harvested at 1 week post-treatment with DMSO (‘Ø’), dabrafenib (‘Da,’ 200 nM), or encorafenib (‘En,’ 200 nM). (H) Cellular morphology imaged by phase contrast of 501mel treated with DMSO, dabrafenib (‘Dab,’ 200 nM), or encorafenib (‘Enco,’ 200 nM) after 72 hr. (I) Representative image and quantification of senescence-associated-β-galactocidase (SA-β-galactocidase) staining after treatment for 72 hr with DMSO, dabrafenib (‘Dab,’ 200 nM), or encorafenib (‘Enco,’ 200 nM). Staining was quantitated manually after blinding samples. Qualitative data represent one of three independent, biological replicates. Quantitative data represent at least three independent experiments. Error bars are S.E.M. Student's t-test compares indicated treatment to matched control. NS, not significant; *p≤1.05; **p≤1.01.

FIG. 3. ERK pathway activity is required for BRAF inhibitor treatment induced growth arrest. (A) Signaling model for rescue experiments. BRAF inhibitors activate RAF signaling, while trametinib and SCH inhibit downstream signaling. At an optimal concentration of both inhibitors, the agonist's output on ERK signaling should be balanced by inhibition with the antagonist. (B) Colony formation assays of trametinib or SCH, titrated across 501mel cells treated with 200 nM encorafenib. Assays were stained with crystal violet, scanned, and quantitated for area on ImageJ. Data are normalized to untreated control. (C) Colony formation assays of trametinib titrated across MeIJUSO treated with either encorafenib (200 nM) or dabrafenib (200 nM). Please note adjusted axis in this panel versus FIG. 3B. Quantitative data represent a least three independent experiments. Error bars are S.E.M.

FIG. 4. Systemic dabrafenib treatment prevents RAS-mutant tumor growth in vivo. (A) IPC 298 cell line xenografts were established in NOD/SCID gamma mice by co-injecting 1 million IPC 298 cells subcutaneously. Daily treatment with vehicle (n=20) or 3.5 mg/kg dabrafenib (n=20) was initiated at ‘Day 0,’ or 7 days post injection. Tumor volume was tracked by caliper measurement, calculated using V=½lw2. Student's t-test compares differences in tumor volumes at sacrifice in both conditions versus vehicle control (B) Representative image of mice from (A) at experimental endpoint. (C) Phospho-ERK western-blotting of tumor lysates from a short-term pharmacodynamic experiment from IPC 298 lysates harvested from mice treated orally with vehicle or 3.5 mg/kg dabrafenib for 5 days. (D) 501mel cell line xenografts were established in athymic nude mice by co-injecting 4 million 501mel cells mixed with 1 million normal dermal fibroblasts (NDF) per site. Mice were treated with vehicle (n=11) or 3.5 mg/kg dabrafenib (n=10), 12 days post injection. As engraftment with 501mel/NDF is inefficient, only tumors with diameters >3 mm at treatment initiation are detailed here. Student's t-test compares differences in tumor volumes at sacrifice in both conditions versus vehicle control. (E) Representative image of mice from (D) at experimental endpoint. (F) Phospho-ERK western-blotting of tumor lysates from a short-term pharmacodynamic experiment in 501mel xenografted mice treated orally with vehicle or 3.5 mg/kg dabrafenib for 5 days. Qualitative data represent one of three independent, biological replicates. Quantitative data represent at least three independent experiments. Error bars are S.E.M. Student's t-test compares indicated treatment to matched control. NS, not significant; *p≤1.05; **p≤1.01.

FIG. 5. The Goldilocks phenomenon for ERK signaling in melanoma. The basic concept is that as ERK pathway-driven tumors, melanomas may have a preferred window of optimal ERK activity which supports proliferation. ERK pathway shutdown likely compromises proliferation, but so does ERK hyperactivation.

FIG. 6. RAS-mutant carcinomas also respond to BRAF inhibitor treatment at submicromolar concentrations. RAS-mutant pancreatic and lung carcinoma cell lines respond to BRAFi. IC50s are calculated in experiments conducted as previously for NRAS-mutant melanoma lines.

FIG. 7. NRAS-mutant melanoma cells upregulate PD-L1 expression following BRAFi exposure. Two sensitive NRAS-mutant melanoma lines (SK-MEL-119 FIG. 7A, 501mel FIG. 7B) were exposed to BRAFi at 100 nM for 3 days and assessed for PD-L1 expression by FACS showing substantial (over 2 to 2.4-fold) upregulation of PD-L1 expression.

FIG. 8. NRAS-mutant melanoma cells upregulate IL-6 expression following BRAFi exposure. Two sensitive NRAS-mutant melanoma lines (SK-MEL-119 FIG. 8A, 501mel FIG. 8B) were exposed to BRAFi at 100 nM for 3 days and assessed for IL-6 expression by FACS.

FIG. 9. Gating strategy of lymphoid (FIG. 9A) and myeloid (FIG. 9B) cells in tumors. Representative plots showing gating strategy followed from CD45+ live cells to distinguish lymphoid and myeloid populations.

FIG. 10. t-SNE plots show segregation of melanoma cells and cells from the tumor microenvironment. The graph represents single cell transcriptomic data obtained on the 10× Genomics platform of a human melanoma specimen and our successful application of analytical tools to discern specific immune and tumor (red) subsets.

FIG. 11. Transplanted NRASQ61R-mutant melanoma cells (C57BL/6) regress when dabrafenib-induced PEASA is combined with anti-PD1 immunotherapy. 1 million melanoma cells were injected in both flanks of C57BL/6 mice and therapy started at 7 days. Anti-PD1 (RMP1-14 thrice weekly; green) and dabrafenib (3.5 mg/kg/day; purple) resulted in partial responses, but progressive disease. The combination results in tumor regression (red). N=6=8 mice per condition across 2 independent trials; mean tumor volume +/−SEM.

FIG. 12 shows control-treated (black) vs. dabrafenib-treated (gray) 501 MEL melanoma cell lines and levels of IL-6 secreted in the media measured after Acute (“A”-24 hours) vs. Chronic (“C”-7 days) exposure as measured in different formats (12-well vs 24-well). Substantial increases in the secretion of this canonical senescence-associated cytokine are reliably measured in all settings, up to 3-fold in magnitude, showing essential features of the ERK-hyperactivation mediated senescence response.

FIGS. 13A to 13C show infiltrating T cells were profiled using FACS following four weeks of treatment with the vehicle (control), dabrafenib alone, anti-PD1 alone and the combination. They show strong activation of key activation markers such as TNF-alpha and IFN-gamma across CD4+(FIG. 13A) and CD8+(FIG. 13B) T-cells essentially only in the combination arm. Additionally, CD8+CD107+ positive cells (FIG> 13C) denote the subpopulation of CD8+ T-cells which have active degranulation activity, indicative of cytolytic activity

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### RAS Mutant Cancers

The disclosed compositions and methods can be used to treat a subject having a Ras mutation, such as an activating Ras mutation. As used herein, the term “activating Ras mutation” refers to any mutation in the Ras oncogene that results in enhanced activity of the Ras polypeptide as assessed by e.g., activation of one or more downstream pathways of Ras. By “enhanced activity” is meant an increase in Ras activity by at least 5% compared to a reference control. Three Ras genes have been identified in the mammalian genome (designated H-ras, K-ras, and N-ras), which acquire cancer cell transformation-inducing properties by single point mutations within their coding sequences. For example, a commonly detected activating Ras mutation found in human tumors is in codon 12 of the H-ras gene in which a base change from GGC to GTC results in a glycine-to-valine substitution in the GTPase regulatory domain of the Ras protein product. This single amino acid change is thought to abolish normal control of Ras protein function, thereby converting it from a normally regulated cellular protein to one that is constitutively active. This de-regulation of normal Ras protein function permits transformation of a cell from a state of normal growth to a state of malignant growth.

The Ras family of small GTPases are frequently mutated in human cancers and are among the most studied oncogenes. Ras is a membrane-bound signaling molecule that cycles between the inactive, GDP-bound state and the active, GTP-bound state. Growth factor receptor signaling promotes GTP loading and activation of Ras, which in turn activates an array of downstream pathways to promote cell proliferation and survival. Among the major Ras effector pathways are the MAP kinase pathway, the PI 3-kinase (PI3K) pathway, RalGDS proteins, phospholipase-Cc and Rac, each of these has been implicated in mediating the tumorigenic effect of the Ras oncogene. Ras GAPs (GTPase activating proteins) inactivate Ras by stimulating its GTP hydrolysis. Oncogenic mutations in Ras are invariably point mutations that either interfere with Ras GAP binding to Ras or directly disrupts Ras GTPase activity, and therefore lock Ras in a constitutively active, GTP-bound state. Oncogenic mutations have been found in all three members of the Ras gene family, KRAS, HRAS and NRAS, with KRAS being the most frequently mutated member. KRAS mutations are found at high frequencies in pancreatic, thyroid, colon, lung, liver cancers and in myelodyspastic syndrome and are correlated with poor prognosis.

Oncogenic H-, K-, and N-Ras arise from point mutations limited to a small number of sites (amino acids 12, 13, 59 and 61). Unlike normal Ras, oncogenic ras proteins lack intrinsic GTPase activity and hence remain constitutively activated. The participation of oncogenic ras in human cancers is estimated to be 30%.

Mutations are frequently limited to only one of the ras genes, and the frequency is tissue- and tumor type-specific. K-ras is the most commonly mutated oncogene in human cancers, especially the codon-12 mutation. While oncogenic activation of H-, K-, and N-Ras arising from single nucleotide substitutions has been observed in 30% of human cancers, over 90% of human pancreatic cancer manifest the codon 12 K-ras mutation. Pancreatic ductal adenocarcinoma, the most common cancer of the pancreas, has a rapid onset and is often resistant to treatment. The high frequency of K-ras mutations in human pancreatic tumors indicates that constitutive Ras activation plays a critical role during pancreatic oncogenesis. Adenocarcinoma of the exocrine pancreas represents the fourth-leading cause of cancer-related mortality in Western countries, Treatment has had limited success and the five-year survival remains less than 5% with a mean survival of 4 months for patients with surgically unresectable tumors. This point mutation can be identified early in the course of the disease when normal cuboidal pancreatic ductal epithelium progresses to a flat hyperplastic lesion, and is considered causative in the pathogenesis of pancreatic cancer.

K-ras mutations are present in 50% of the cancers of colon and lung. In cancers of the urinary tract and bladder, mutations are primarily in the H-ras gene. N-ras gene mutations are present in 30% of leukemia and liver cancer. Approximately 25% of skin lesions in humans involve mutations of the Fla-Ras (25% for squamous cell carcinoma and 28% for melanomas). 50-60% of thyroid carcinomas are unique in having mutations in all three genes.

Constitutive activation of Ras can be achieved through oncogenic mutations or via hyper-activated growth factor receptors such as the EGFRs. Elevated expression and/or amplification of the members of the EGFR family, especially the EGFR and HER2, have been implicated in various forms of human malignancies. In some of these cancers (including pancreas, colon, bladder, lung), EGFR1HER2 overexpression is compounded by the presence of oncogenic Ras mutations. Abnormal activation of these receptors in tumors can be attributed to overexpression, gene amplification, constitutive activation mutations or autocrine growth factor loops. For growth factor receptors, especially the EGFRs, amplification or/and overexpression of these receptors frequently occur in the cancers of the breast, ovary, stomach, esophagus, pancreatic, lung, colon neuroblastoma.

### BRAFi

Any selective BRAF inhibitor (BRAFi) can be used in the disclosed compositions and methods. As used herein, “selective BRAFi” refers to any agent capable of inhibiting BRAF expression or activity in a subject without also inhibiting another RAF, such as CRAF.

BRAF inhibitors induce allosteric structural rearrangements, which “lock” their target kinases in discrete conformations and resemble inactive or active kinase states of the αC-helix and DFG motif. These conformations broadly classify inhibitors as type I (αC-helix-IN/DFG-IN), type II (αC-helix-IN/DFG-OUT), or type I 1/2 (αC-helix-OUT/DFG-IN). In some embodiments, the BRAFi is one that shifts the BRAF αC helix toward an inactive conformation, such as vemurafenib, PLX4720, or AR004549. In some embodiments, the BRAFi favors the active orientation of the αC helix, such as GDC-0879.

Agianian B, et al. J Med. Chem. 2019 61:5775-5793 provides a review of known type I, II, and I 1/2 BRAF inhibitors, which is incorporated by reference for these BRAFi. Examples of BRAF inhibitors include Vemurafenib (Zelboraf), dabrafenib (Tafinlar), and encorafenib (Braftovi).

Vemurafenib (Zelboraf, PLX4032) (shown below) is a V600 mutant B-Raf inhibitor approved by the FDA for the treatment of late-stage melanoma.

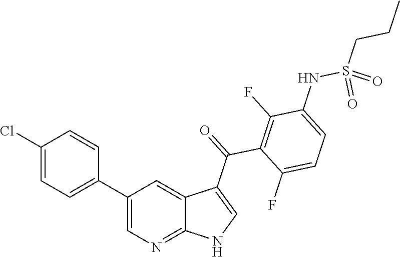 [SMILES: CCCS(=O)(=O)Nc1ccc(F)c(C(=O)c2c[nH]c3ncc(-c4ccc(Cl)cc4)cc23)c1F]

Unlike BAY43-9006, which inhibits the inactive form of the kinase domain, Vemurafenib inhibits the active “DFG-in” form of the kinase, firmly anchoring itself in the ATP-binding site. By inhibiting only the active form of the kinase, Vemurafenib selectively inhibits the proliferation of cells with unregulated B-Raf, normally those that cause cancer. Since Vemurafenib only differs from its precursor, PLX4720, in a phenyl ring added for pharmacokinetic reasons, PLX4720's mode of action is equivalent to Vemurafenib's. PLX4720 has good affinity for the ATP binding site partially because its anchor region, a 7-azaindole bicyclic, only differs from the natural adenine that occupies the site in two places where nitrogen atoms have been replaced by carbon. This enables strong intermolecular interactions like N7 hydrogen bonding to C532 and N1 hydrogen bonding to Q530 to be preserved. Excellent fit within the ATP-binding hydrophobic pocket (C532, W531, T529, L514, A481) increases binding affinity as well. Ketone linker hydrogen bonding to water and difluoro-phenyl fit in a second hydrophobic pocket (A481, V482, K483, V471, 1527, T529, L514, and F583) contribute to the exceptionally high binding affinity overall. Selective binding to active Raf is accomplished by the terminal propyl group that binds to a Raf-selective pocket created by a shift of the αC helix. Selectivity for the active conformation of the kinase is further increased by a pH-sensitive deprotonated sulfonamide group that is stabilized by hydrogen bonding with the backbone peptide NH of D594 in the active state. In the inactive state, the inhibitor's sulfonamide group interacts with the backbone carbonyl of that residue instead, creating repulsion. Thus, Vemurafenib binds preferentially to the active state of B-Raf's kinase domain.

Dabrafenib (Tafinlar, GSK2118436) (shown below) is a single agent treatment for patients with BRAF V600E mutation-positive advanced melanoma.

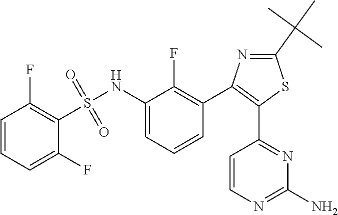 [SMILES: CC(C)(C)c1nc(-c2cccc(NS(=O)(=O)c3c(F)cccc3F)c2F)c(-c2ccnc(N)n2)s1]

Clinical trial data demonstrated that resistance to dabrafenib and other BRAF inhibitors occurs within 6 to 7 months. To overcome this resistance, the BRAF inhibitor dabrafenib was combined with the MEK inhibitor trametinib. On Jan. 8, 2014, the FDA approved this combination of dabrafenib and trametinib for BRAF V600E/K-mutant metastatic melanoma. On May 1, 2018, the FDA approved the combination dabrafenib/trametinib as an adjuvant treatment for BRAF V600E-mutated, stage III melanoma after surgical resection based on the results of the COMBI-AD phase 3 study, making it the first oral chemotherapy regimen that prevents cancer relapse for node positive, BRAF-mutated melanoma.

Encorafenib (Braftovi) (shown below) is a small molecule BRAF inhibitor that targets key enzymes in the MAPK signaling pathway.

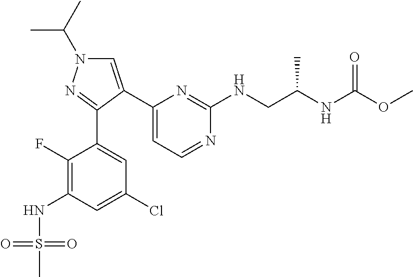 [SMILES: COC(=O)N[C@@H](C)CNc1nccc(-c2cn(C(C)C)nc2-c2cc(Cl)cc(NS(C)(=O)=O)c2F)n1]

This pathway occurs in many different cancers including melanoma and colorectal cancers. In June 2018 it was approved by the FDA in combination with binimetinib for the treatment of patients with unresectable or metastatic BRAF V600E or V600K mutation-positive melanoma. Encorafenib acts as an ATP-competitive RAF kinase inhibitor, decreasing ERK phosphorylation and down-regulation of CyclinD1. This arrests the cell cycle in G1 phase, inducing senescence without apoptosis. Therefore it is only effective in melanomas with a BRAF mutation, which make up 50% of all melanomas. The plasma elimination half-life of encorafenib is approximately 6 hours, occurring mainly through metabolism via cytochrome P450 enzymes.

Sorafenib (BAY43-9006, Nexavar) (shown below) is a V600E mutant B-Raf and C-Raf inhibitor approved by the FDA for the treatment of primary liver and kidney cancer. Bay43-9006 disables the B-Raf kinase domain by locking the enzyme in its inactive form.

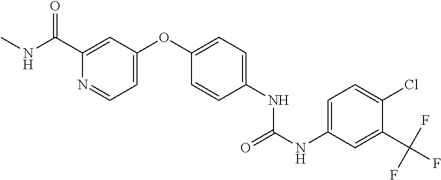 [SMILES: CNC(=O)c1cc(Oc2ccc(NC(=O)Nc3ccc(Cl)c(C(F)(F)F)c3)cc2)ccn1]

The inhibitor accomplishes this by blocking the ATP binding pocket through high-affinity for the kinase domain. It then binds key activation loop and DFG motif residues to stop the movement of the activation loop and DFG motif to the active conformation. Finally, a trifluoromethyl phenyl moiety sterically blocks the DFG motif and activation loop active conformation site, making it impossible for the kinase domain to shift conformation to become active. The distal pyridyl ring of BAY43-9006 anchors in the hydrophobic nucleotide-binding pocket of the kinase N-lobe, interacting with W531, F583, and F595. The hydrophobic interactions with catalytic loop F583 and DFG motif F595 stabilize the inactive conformation of these structures, decreasing the likelihood of enzyme activation. Further hydrophobic interaction of K483, L514, and T529 with the center phenyl ring increase the affinity of the kinase domain for the inhibitor. Hydrophobic interaction of F595 with the center ring as well decreases the energetic favorability of a DFG conformation switch further. Finally, polar interactions of BAY43-9006 with the kinase domain continue this trend of increasing enzyme affinity for the inhibitor and stabilizing DFG residues in the inactive conformation. E501 and C532 hydrogen bond the urea and pyridyl groups of the inhibitor respectively while the urea carbonyl accepts a hydrogen bond from D594's backbone amide nitrogen to lock the DFG motif in place. The trifluoromethyl phenyl moiety cements the thermodynamic favorability of the inactive conformation when the kinase domain is bound to BAY43-9006 by sterically blocking the hydrophobic pocket between the αC and αE helices that the DFG motif and activation loop would inhabit upon shifting to their locations in the active conformation of the protein.

In some embodiments, the disclosed BRAFi is any agent that inhibits BRAFi and activates ERK and/or RAS signaling. BRAF inhibitors PLX8394 and PLX7904, dubbed as “paradox breakers”, were developed to inhibit V600 mutated oncogenic BRAF without causing paradoxical MAPK pathway activation. Therefore, in some embodiments, the BRAFi is not PLX8934 or PLX7904.

### ERK/RAS Activator

Small molecules that activate ERK and RAS signaling are disclosed, for example, in Abbott J R, et al. J. Med. Chem. 2018, 61, 6002-6017 and Howes, J E, et al. Mol Cancer Ther 2018; 17:1051-1060, which are incorporated by reference in their entireties for the teaching of these agents.

Howes J E, et al. Mol Cancer Ther. 2018 17(5):1051-1060 describes a small molecule that can activates RAS and ERK by targeting SOS1, which is incorporated by reference in its entirety for the teaching of this agent. Abbott J R, et al. J. Med. Chem. 2018 61:6002-6017 describes aminopiperidone indoles that activate SOS1 and modulate RAS signaling, which is incorporated by reference in its entirety for the teaching of this molecules.

### Immunotherapy

Immunotherapy is a form of oncologic treatment directed towards enhancing the host immune system against cancer. In recent years, manipulation of immune checkpoints or pathways has emerged as an important and effective form of immunotherapy. Immunotherapy also includes chimeric monoclonal antibodies and antibody drug conjugates that target malignant cells and promote their destruction. Genetically modified T cells expressing chimeric antigen receptors are able to recognize specific antigens on cancer cells and subsequently activate the immune system. Native or genetically modified viruses with oncolytic activity can destroy malignant cells and increase anti-tumor activity in response to the release of new antigens and danger signals as a result of infection and tumor cell lysis. Vaccines are also being explored, either in the form of autologous or allogenic tumor peptide antigens, genetically modified dendritic cells that express tumor peptides, or even in the use of RNA, DNA, bacteria, or virus as vectors of specific tumor markers.

The disclosed immunotherapy can therefore be a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the immunotherapy involves tumor-directed monoclonal antibodies. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Of particular interest are the recently developed bispecific antibodies that combine antigen-binding specificities on tumor cells and effector immune cells. Among these, bispecific T cell engager (BiTE) and dual-affinity re-targeting (DART) are particularly attractive. BiTEs recombinantly link the four variable domains of heavy and light chains with a flexible linker peptide allowing to bypass MHC/peptide recognition and co-stimulation and also to bring effector cells and target cells close together to form cytolytic synapses. DART consists of a diabody that separates variable domains of heavy and light chains of the two antigen-binding specificities on two separate polypeptide chains stabilized through a C-terminal disulfide bridge which acts as a linker.BiTEs simultaneously target two different antigens and thus target two different mediators and pathways. For example, CEA CD3 TCB (RG7802, R06958688) is an IgG1 BiTE that simultaneously binds carcinoembryonic antigen (CEA) on tumor cells and CD3 on T cells to increase tumor-infiltrating lymphocytes (TIL) activation, infiltration, and expression of PD-1/PD-L1. Blinatumomab is another BiTE that binds CD3 on T cells as well as CD19 on malignant B cells. BAY2010112 (AMG212, MT112) and MOR209/ES414 are prostate-specific membrane antigen (PSMA)/CD3 BiTEs. MGD009 is a humanized DART protein that binds both T cells and tumor-associated B7-H3. AFM13 is a tetravalent bispecific antibody that is directed against CD30 and CD16A, this latter found over natural killer (NK) cells.

Antibody drug conjugates (ADCs), an emerging therapeutic approach in oncology, combine a monoclonal antibody with a high selectivity for specific targets with a cytotoxic agent. Microtubule inhibitors or DNA-damaging chemotherapeutic agents are the two main cytotoxic agents used in ADCs. An ideal antigen is one that is overexpressed by malignant cells with very limited or no expression by normal tissue. For example, nectin-4 is often overexpressed in bladder, breast, lung, and pancreatic cancer, and thus, ACDs against this peptide are indicated in these malignancies. Similarly, folate receptor alpha is more often expressed by ovarian and endometrial carcinomas, and CEA cell adhesion molecule (CEACAM) 5 is commonly found on CRC. ABBV-399 is an ADC composed of an anti-c-Met antibody (ABT-700) conjugated to a microtubule inhibitor (monomethyl auristatin E). Glembatumumab vedotin (GV, CDX-011) is an ADC that contains an antibody that targets glycoprotein non-metastatic b (gpNMB), a transmembrane glycoprotein usually overexpressed in melanoma and other tumors, conjugated to monomethyl auristatin E. Losatuxizumab vedotin (ABBV-221), an ADC that targets EGFR. Mirvetuximab soravtansine (IMGN853) is an ADC containing the tubulin inhibitor (maytansinoid) DM4, targeting the folate receptor alpha (FRα). Enfortumab vedotin (ASG-22CE; ASG-22ME), an ADC that targets nectin-4. Sacituzumab govitecan (IMMU-132) is an ADC against Trop-2 antigen expressed in many solid tumors and carrying the topoisomerase inhibitor, SN-38. Inotuzumab ozogamicin (InO/CMC-544) is a humanized ADC directed against CD22, coupled to a DNA breaking calicheamicin. Labetuzumab govitecan (IMMU-130) is an ADC that targets CEACAM 5 which is expressed by >80% of CRC. Lorvotuzumab mertansine (IMGN901), an ADC against CD56 conjugated to the tubulin inhibitor DM1. Rovalpituzumab tesirine (Rova-T) is an ADC that targets delta-like protein 3 (DLL3). ADCT-301 is the first ADC against CD25, a receptor for IL-2 often found on hematological tumors. TAK-264 (MLN0264), a novel ADC that targets guanylyl cyclase C (GCC). T-DM1 (Kadcyla), an ADC consisting of trastuzumab (T) and a microtubule inhibitor (DM1), was the first ADC approved by the FDA to use in solid tumors.

Chimeric antigen receptor (CAR) T cells are typically genetically engineered T cell receptors with an antibody-based extracellular domain that specifically recognizes a tumor antigen, a transmembrane portion, and an intracellular domain that activates the T cell. By antigen-specific recognition in a MHC-independent manner, CAR T cells are activated in vivo through phosphorylation of immune receptor tyrosine-based activation motifs (ITAMs) leading to cytokine secretion, T cell proliferation, and antigen-specific cytotoxicity. CAR T cells are produced by inserting specific CAR genes via viral vectors into autologous or allogeneic T cells. New-generation CARs have two or more co-stimulatory domains (e.g., 4-1BB, OX 40) that boost the stimulatory signal. Anti-CD19 CAR T cells were recently FDA-approved for B-ALL in pediatric and young adult population. Other CARs rely on the ligand of the receptor of interest rather than on an antibody. T4 immunotherapy uses genetically engineered T cells that co-express two CARs, T1E28z that targets ErbB dimers, and 4a13 that binds IL-4 and promotes T cell expansion. 19-28z CAR (JCAR015) consists of a single-chain murine antibody against human CD19 (expressed by B cell malignancies) fused with the transmembrane and cytoplasmic domains of the human CD28 co-stimulatory molecule. CAR-T cells have also been developed that target GPC3, CD133, BCMA, CD138, CD30, IL13Ra2, NKG2D, NKR-2, and mesothelin.

In contrast to CAR T cell therapy, T cell receptor (TCR) gene-modified T cell therapy functions by targeting the surface antigens of tumor cells to specifically recognize intracellular tumor antigens presented by HLA molecules. Currently, genes encoding TCRs that are specific for a variety of tumor antigens such as MART-1, gp100, p53, NY-ESO-1, MAGE-A3, and MAGE-A4 have been studied as therapeutic targets for TCR gene-modified T cell therapy in clinical trials for melanoma, lung cancer, and breast cancer patients.

Adoptive cell therapy that utilizes endogenous tumor-infiltrating lymphocytes (TIL), which are expanded in vitro from a surgically resected tumor and then re-infused back into the patient, has demonstrated a 20% complete response lasting beyond 3 years in patients with stage IV melanoma. TILs are naturally occurring T cells in the host able to recognize tumor antigens. This likely explains the highly specific anti-tumor responses and the relatively low toxicity of TILs in comparison with TCR gene-modified T cell therapy and CAR T cell therapy. In addition, TILs are heterogeneous in their specificity which represents an important advantage for impeding immunologic escape. Furthermore, TIL therapy bypasses the limitation identifying specific tumor antigens or the patient's HLA type.

Native or genetically modified viruses are a new therapeutic approach within the immunotherapy spectrum. The mechanisms of action of oncolytic viruses are not fully elucidated but likely depend on viral replication within tumor cells, induction of primary cell death, interaction with tumor cell antiviral elements, and initiation of innate and adaptive anti-tumor immunity. A variety of native and genetically modified viruses have been developed as oncolytic agents. Of note, these viruses selectively infect malignant cells due to the lack of adequate function of anti-viral mechanisms. Though many viruses have been considered, the most widely studied to date include herpes simplex virus type 1 (HSV-1), coxsackievirus, reovirus, and adenovirus. Talimogene laherparepvec (T-VEC; Imlygic) is the first oncolytic virus approved by the FDA for its use in melanoma. Coxsackievirus A21 (CVA21-CAVATAK) preferentially infects tumors that express ICAM-1. Pelareorep (Reolysin) is a strain of reovirus serotype-3 which has shown in vitro and in vivo activity against many cancers and synergistic activity with concomitant use of microtubule-targeting drugs.

Therapeutic vaccines are designed to increase immune response against malignant cells by enlarging antigen-specific T cell from endogenous T cell repertoire. Depending on its composition, vaccines can be classified into tumor cell vaccines (autologous/allogenic), genetic vaccines (DNA/RNA/viral/bacterial), dendritic cell (DC) vaccines, and protein/peptide vaccines.

In some embodiments, the immunotherapy comprises cytokine gene therapy. IL-12 has been considered a good option for immunotherapy given its potent anti-tumor effect. Ad-RTS-hIL-12 is a replication-incompetent adenovirus engineered to express IL-12. By default, IL-12 expression by this virus is “off,” but with the use of veledimex, gene is activated and IL-12 production is started. IL-2 enhances the immune system through the IL-2 receptor (IL-2R). NKTR-214 is an engineered cytokine that specifically stimulates IL-2R.

### Pharmaceutical Composition

Also disclosed is a pharmaceutical composition comprising a BRAFi and an immunotherapy in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Methods of Treatment

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the composition is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule containing lenalidomide and/or erythropoietin administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

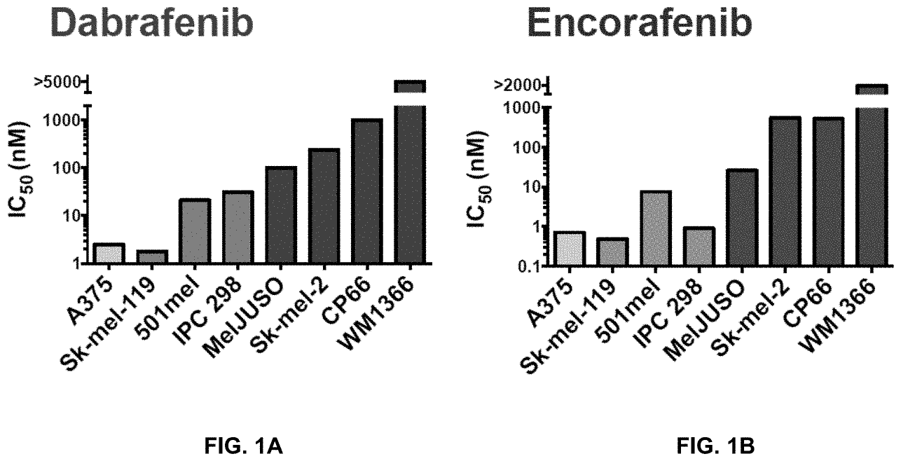

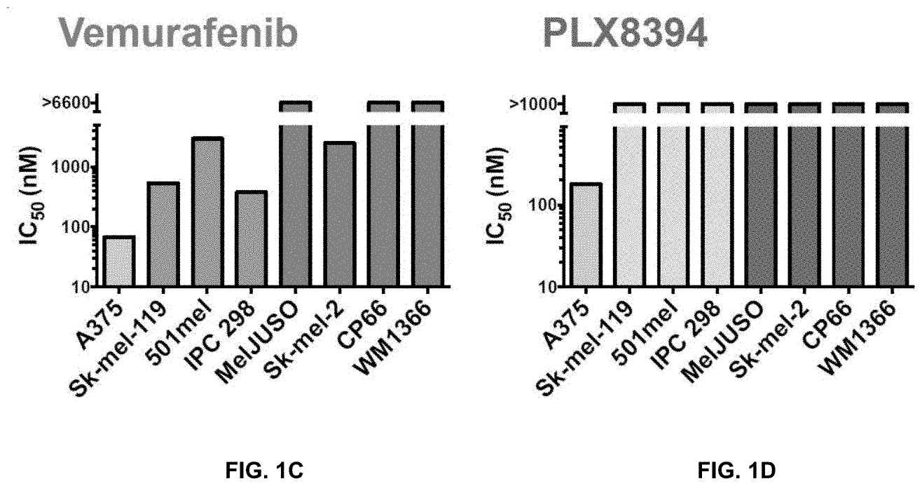

While oncogene-induced senescence due to overexpressed mutant RAS is an important tumor suppression mechanism, multiple variants of senescence exist (Ito, Y., et al. Trends Cell Biol 27:820-832 (2017); Lasry, A. & Ben-Neriah, Y. Trends Immunol 36:217-28 (2015); Ewald, J. A., et al. J Natl Cancer Inst 102:1536-46 (2010); Mooi, W. J. & Peeper, D.S. N Engl J Med. 355:1037-46 (2006)). With this in mind, experiments were conducted to determine whether paradoxical ERK activation, as driven by BRAFi exposure in the context of RAS-mutant cancers, could cause elevation of ERK pathway signaling to the point of causing senescence-like growth arrest akin to oncogene-induced senescence. To determine the effects of BRAFi treatment on RAS-mutant melanomas, a panel of four BRAFi were screened against eight RAS-mutant melanoma cell lines. the FDA-approved BRAFi vemurafenib, dabrafenib, encorafenib (Dummer, R., et al. J Clin Oncol 31(2013)), and the ‘paradox-breaker PLX8394, which was designed to elicit no paradoxical ERK activation (Zhang, C., et al. Nature 526:583-6 (2015)) were tested. Indeed, dabrafenib and encorafenib significantly inhibited the growth of four ‘BRAFi sensitive’ RAS-mutant cell lines with IC₅₀ below 100 nM (FIGS. 1-4, Tables 1-2), comparable to responses of BRAF-mutant cell lines. This suggested that the observations in RAS-mutant cell lines could be relevant to in-vivo endpoints (Adelmann, C. H., et al. Oncotarget 7:30453-60 (2016)). Notably, the paradox-breaker PLX8394 never showed activity against RAS mutant cell lines. This key difference suggested that dabrafenib, encorafenib, and vemurafenib could be driving growth inhibition through ERK activation (Zhang, C., et al. Nature 526:583-6 (2015)).

TABLE 1
BRAF inhibitor treatment blocks the growth of RAS-mutant melanoma
cell lines at sub-micromolar concentrations. This table summarizes the
IC₅₀ of the melanoma cell lines tested in FIG. 1.

| | RAS | Vemurafenib | Dabrafenib | Encorafenib | PLX8394 |
|---|---|---|---|---|---|
| Cell Line | Genotype | IC₅₀ (nM) | IC₅₀ (nM) | IC₅₀ (nM) | IC₅₀ (nM) |
| A375 | WT | 68 | 2.5 | 0.71 | 180 |
| | (BRAFV600E) | | | | |
| ICP 298 | Q61L | 380 | 31 | 0.9 | >1000 |
| Sk-mel-119 | Q61R | 540 | 1.8 | 0.49 | >1000 |
| Sk-mel-2 | Q61R | 2500 | 240 | 550 | >1000 |
| 501mel | G12D | 3000 | 21 | 7.5 | >1000 |
| GAK | Q61K | 5300 | 700 | 1600 | >1000 |
| MelJUSO | Q61L | >6600 | 100 | 26 | >1000 |
| | (HRASG13D) | | | | |
| CP66 | Q61K | >6600 | 1000 | 520 | >1000 |
| WM1366 | Q61L | >6600 | >5000 | >2000 | >1000 |

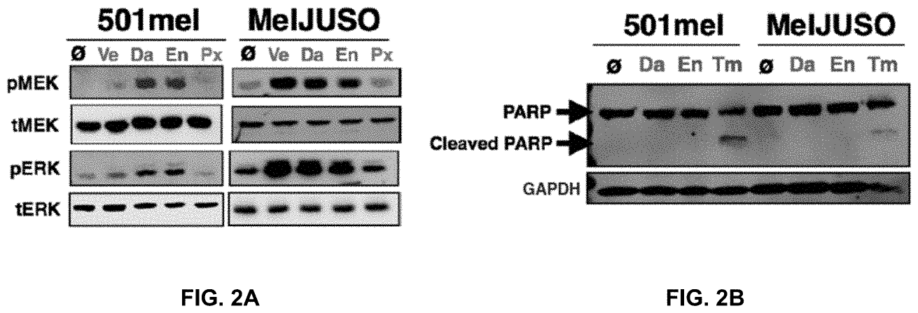

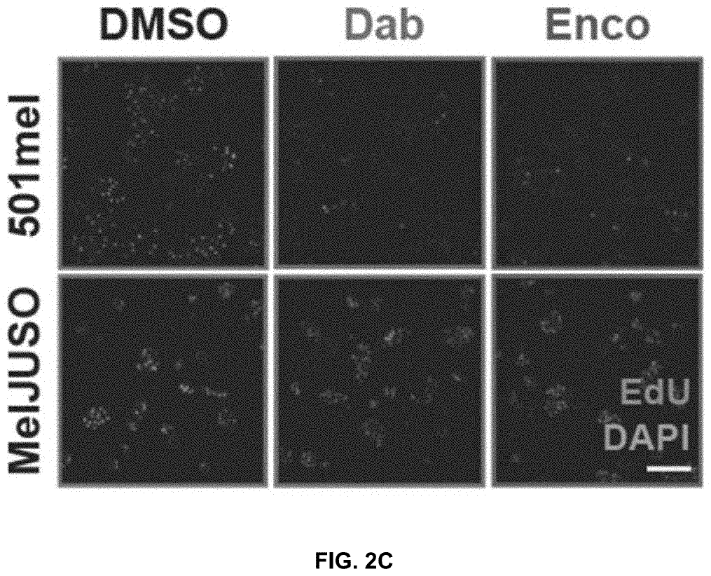

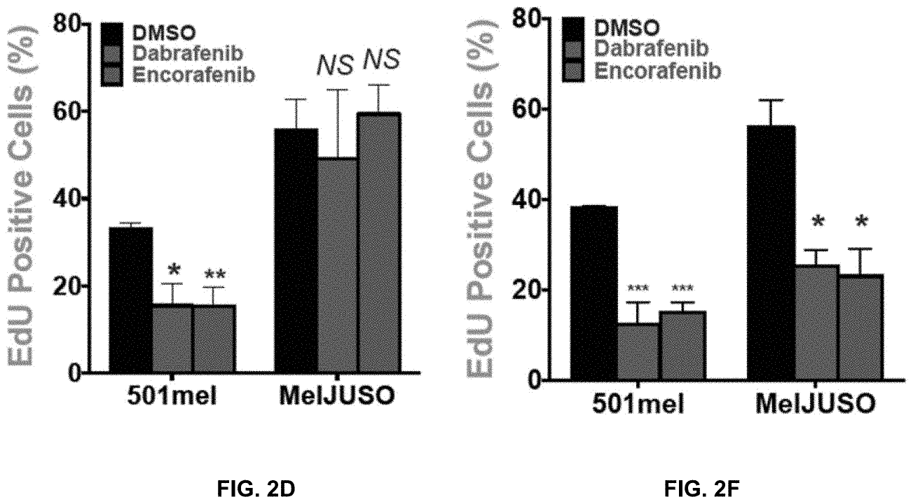

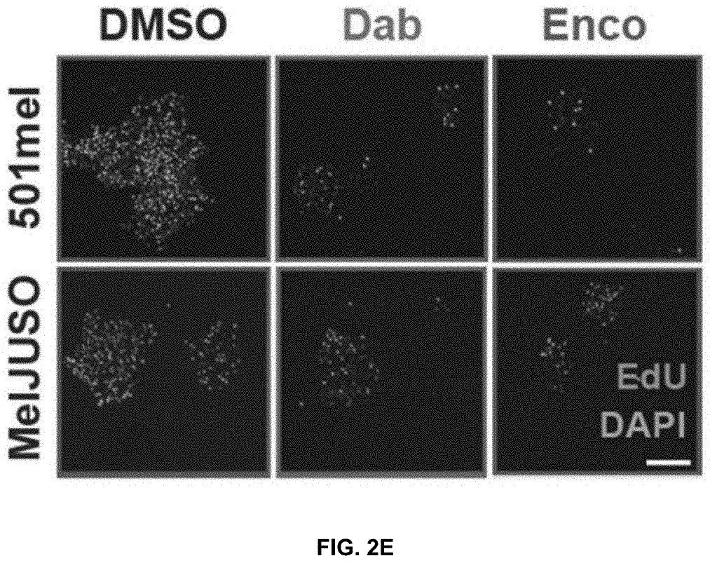

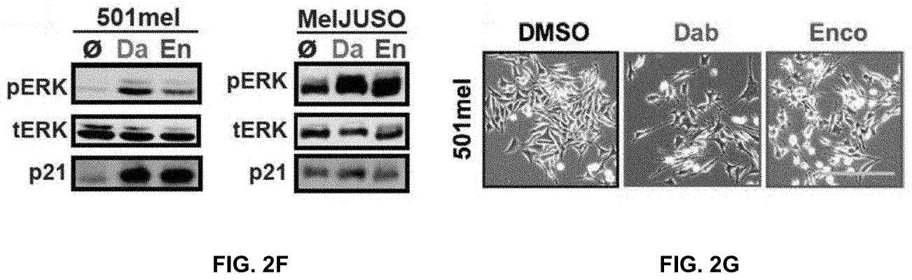

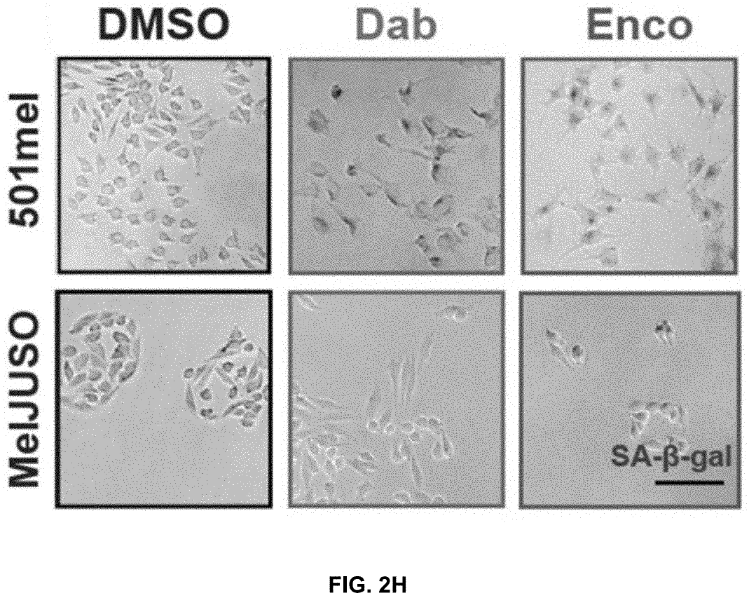

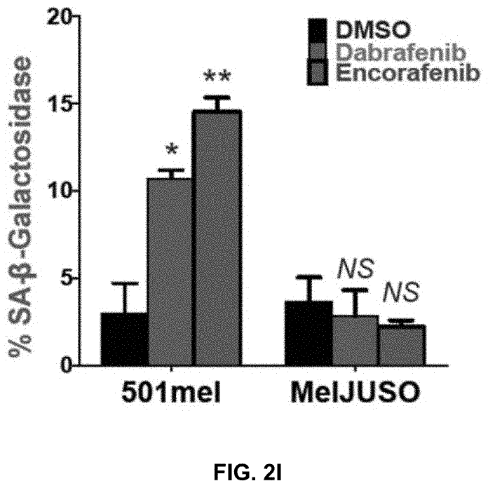

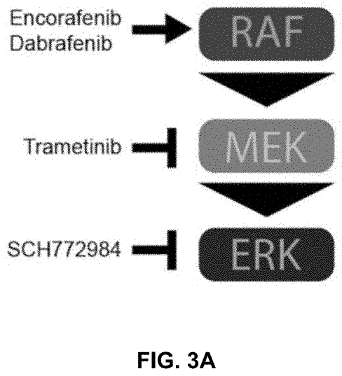

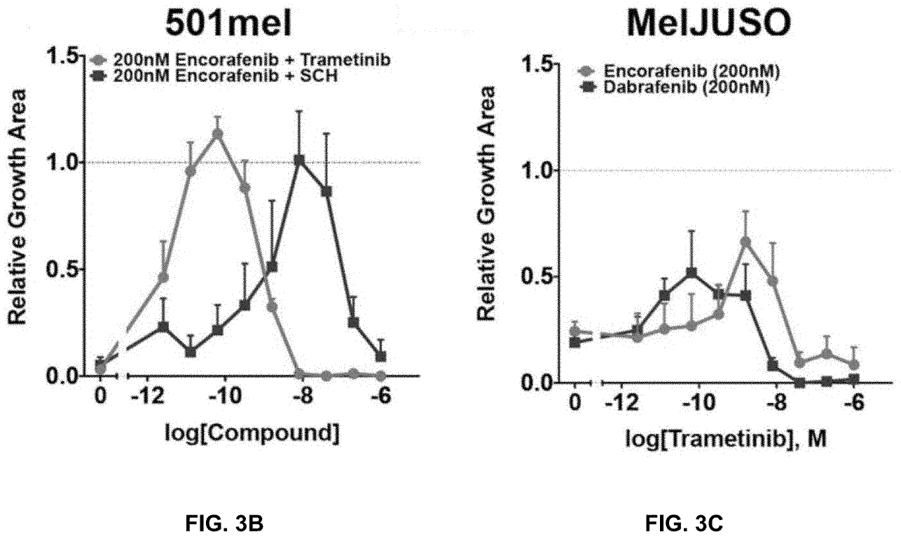

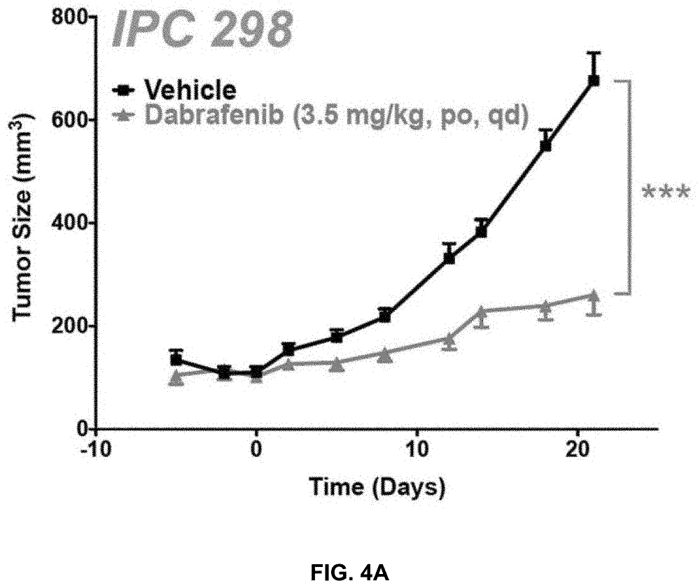

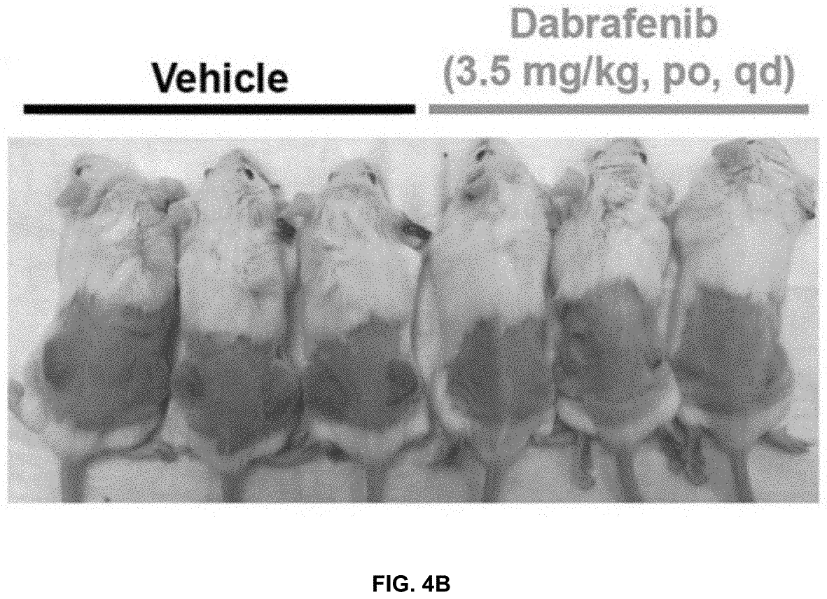

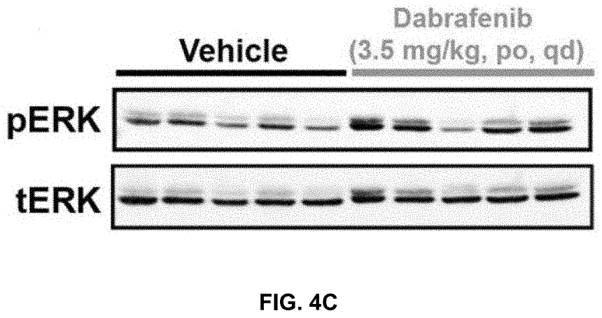

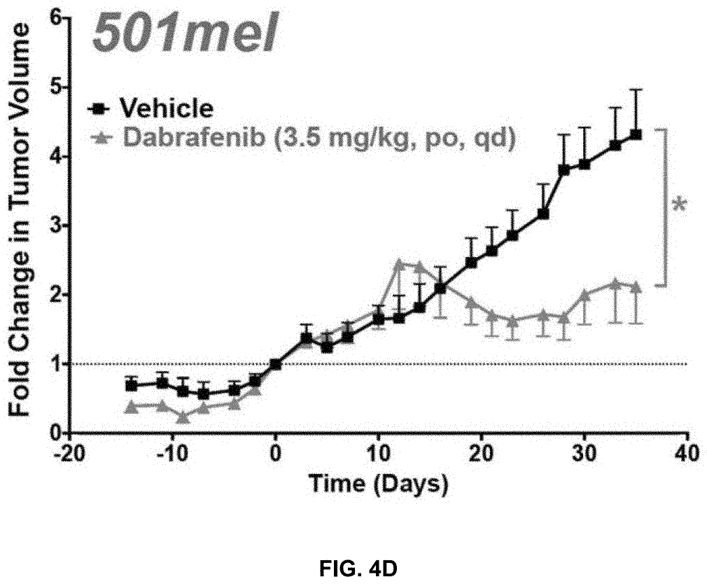

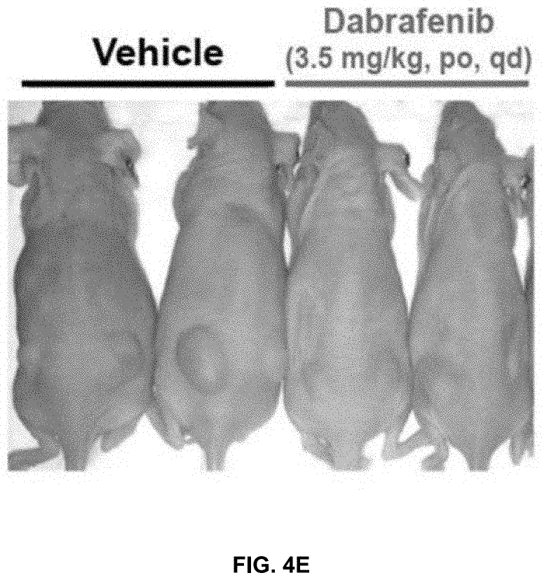

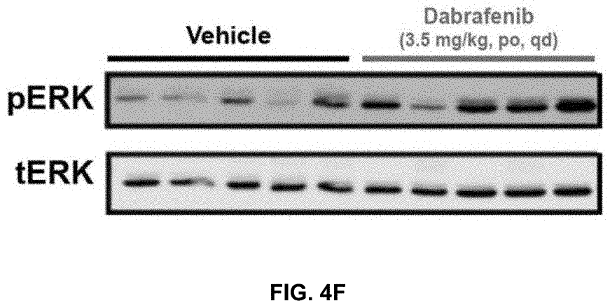

BRAFi strongly increased phosphorylated MEK and ERK, p21 expression, and senescence-associated-β-galactosidase (SA-β-Gal) activity (FIG. 2). To test if ERK activation was specifically responsible for growth inhibition in BRAFi-treated cell lines, elevated ERK signaling levels were progressively decreased in BRAFi-treated 501mel cells by co-treating with increasing doses of MEK and ERK inhibitors. Consistent with ERK hyperactivation being necessary for the arrest due to BRAFi in 501mel, increasing ERK pathway inhibition essentially completely restored proliferation to dabrafenib-treated 501mel cells before blocking growth at higher levels (FIG. 3). This presumably occurred as ERK signaling decreased below physiologically normal levels. In-vivo treatment of xenografted human melanoma lines resulted in tumor stabilization over 35 days, with hyperactivation of ERK activity in tumors evident by western (FIG. 4).

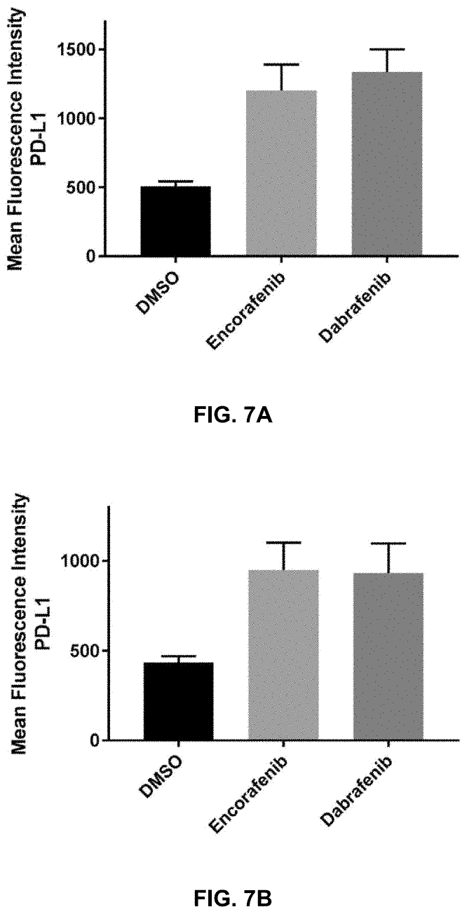

Because senescence is associated with a specific inflammatory gene expression signature, RNAseq was used to characterize responsive NRAS-mutant cell lines (IPC298, SK-MEL-119) following BRAFi exposure. There was upregulation of PD-L1 expression and TNFα/IFNγ gene signatures suggestive of a pro-inflammatory response (FIG. 7). Using a transplantable C57BL/6 mouse model of NRAS^{Q61R}-mutant melanoma (Burd, C. E., et al. Cancer Discov 4:1418-29 (2014)), tumor regression can be observed only in the anti-PD1-dabrafenib combination (FIG. 11), suggesting this approach may sensitize tumors to ICB. These data provide strong rationale for the ability to delineate the mechanisms of pharmacological ERK-activation induced senescence-like arrest (PEASA), elucidate determinants of response and resistance, identify optimal combinations with ICB, and address disease refractory to ICB.

Although 30% of human cancers are driven by mutant RAS, few solutions have been proposed. Historically, oncogenic pathways have been regarded as key targets for inhibition. However, it is unknown whether modulation of these pathways in other ways can be useful. Interestingly, many BRAF inhibitors (BRAFi) paradoxically activate ERK in BRAF-wild-type cells (Adelmann, C. H., et al. Oncotarget 7:30453-60 (2016); Heidorn, S. J., et al. Cell 140:209-21 (2010); Hatzivassiliou, G., et al. Nature 464:431-5 (2010); Poulikakos, P. I., et al. Nature 464:427-30 (2010); Karreth, F. A., et al. Mol Cell 36:477-86 (2009)). Inducing paradoxical ERK activation in RAS-mutant cancers may therefore elevate ERK signaling to a degree so as to induce senescence-like proliferation arrest akin to oncogene-induced senescence.

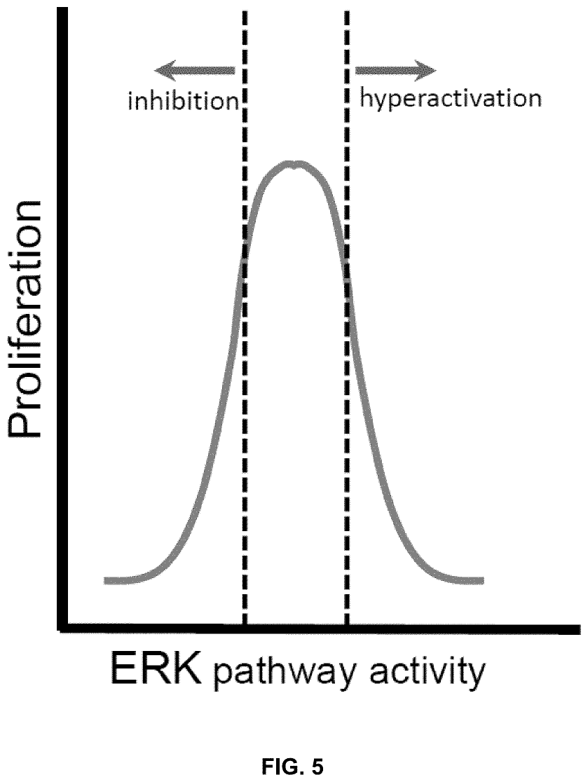

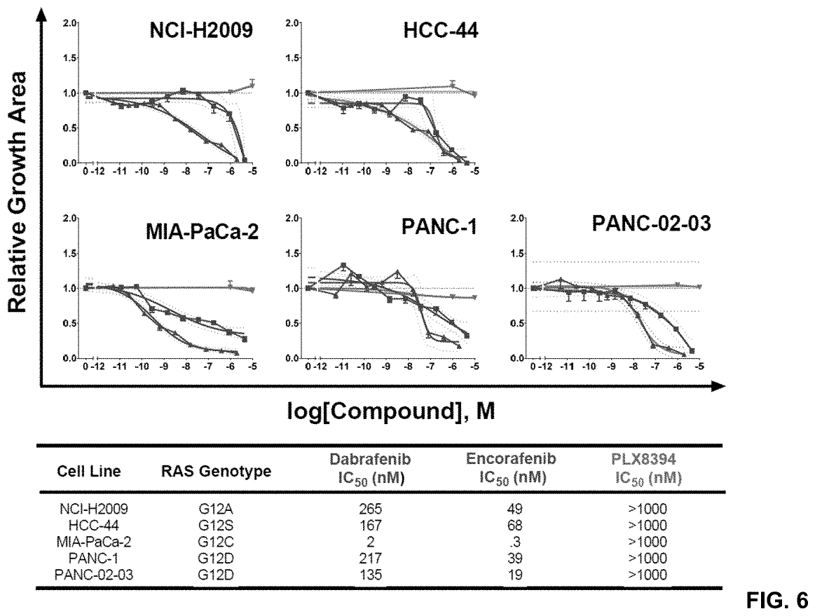

The exploration of this is strongly supported by data in both cultured NRAS-mutant melanoma and KRAS-mutant carcinoma lines and in-vivo. The reason that the effects of BRAFi on RAS-mutant lines have not been previously reported is that PEASA occurs over 1-2 weeks, longer than most cell-based inhibitor assays typically performed at 72h. Published outcomes of patients with definitively RAS-mutant melanomas and carcinomas treated with BRAFi are lacking. The disclosed evidence overwhelmingly shows in more than 8 definitively genotyped cell lines of diverse lineages, that PEASA causes arrest at clinically-relevant doses in culture and in-vivo (FIGS. 1-6, Table 1) suggesting for some tumors, a small window of optimal ERK activation is needed (FIG. 5). Furthermore, the complete lack of response using a BRAFi incapable of paradoxical ERK activation, and the dependence of the arrest on hyperactive ERK strengthens this significantly. PEASA is not universally effective; however, that is true of all therapies and it merits further study.

As disclosed herein, PEASA is an effective anti-tumor strategy through induction of tumor cell proliferation arrest, creation of an inflammatory microenvironment, and sensitization of tumors to ICB.

### Example 2: Identify the Mechanism of Pharmacologic ERK Activation-Induced Senescence-Like Arrest (PEASA)

The above data demonstrate that in a large panel of RAS-mutant cell lines, BRAFi induce long-term growth arrest with features of senescence (FIGS. 1-6). However, it is clear that RAS-mutant cells are neither universally nor equally sensitive. Experiments are conducted to determine if these differences in response are driven by specific genetic differences and different signaling responses. The TP53 and INK4A status of all the cell lines used in the study have been assembled (Table 2).

Although it is unclear whether response is exclusively associated with specific genotypes, these features are likely to be important. In addition to periodic routine STR-based cell authentication, the accuracy of these genotypes (Table 2) is verified using targeted exome sequencing (Qiaseq Cancer Panel; Moffitt Genomics Core). Further sensitivity correlations with additional mutations can then be assessed.

Baseline levels of pERK and its canonical target phospho-p90^{RSK} is measured and correlated with sensitivity. Although most of the NRAS-mutant melanoma and KRAS-mutant lung cancer cell lines tested exhibit submicromolar sensitivity to BRAFi, the mechanism is incompletely characterized. Therefore, experiments are conducted to test for induction of p53, p21, p27, p16INK4A, p19ARF, and H3K9me3 to systematically characterize the growth arrest profile of these cells following exposure to dabrafenib and encorafenib (Hennessey, R. C., et al. Pigment Cell Melanoma Res 30:477-487 (2017)). The trimethylated histone H3K9 is upregulated in chemotherapy-induced senescence (Yu, Y., et al. Cancer Cell 33:322-336 e8 (2018); Webster, M. R., et al. Pigment Cell Melanoma Res 28:184-95 (2015)). After the upregulation of H3K9me3 in treated lines is confirmed, CHIPseq is performed to identify H3K9me3 bound DNA sequences. The sensitive SK-MEL-119, IPC298, H2009 lines and relatively resistant CP66, WM1366, A549 lines are treated with 100 nM dabrafenib or encorafenib for 6-24 hours to examine acute responses and use western blots to measure the expression of the above mediators.

To complement this, a discovery-based RNAseq and microRNAseq is performed on sensitive SK-MEL-119, IPC298, H2009 lines and relatively resistant CP66, WM1366, A549 lines. The analysis focuses on GSEA to identify the most relevant pathways that correlate with sensitivity, differentially expressed genes and miRNAs between control and drug-treated cells. This data is mapped to the H3K9me3 CHIPseq data to identify changes regulated by H3K9 trimethylation. These experiments identify key transcriptional drivers of PEASA. This data also impacts identification of the core inflammatory signatures generated by PEASA.

Experiments are also conducted to determine if RAS-mutant cells adapt following exposure to BRAFi to downregulate ERK activity to as to regain proliferative potential. Candidate regulators include the SPRY, SPRED, and DUSP families of proteins which are known to downregulate MAPK signaling by regulating RAS, RAF, MEK and ERK activation (Cabrita, M.A. & Christofori, G. Angiogenesis 11:53-62 (2008); Bundschu, K., et al. Bioessays 29:897-907 (2007); McClatchey, A. I. & Cichowski, K. Genes Dev 26:515-9 (2012); Petti, C., et al. Cancer Res 66:6503-11 (2006); Sasaki, A., et al. Nat Cell Biol 5:427-32 (2003)). In BRAF/NRAS-double mutant melanomas, SPRY4 is induced and downregulates ERK signaling to a level compatible with continued proliferation (Kumar, R., et al. Oncogene 38:3504-3520 (2019)). Finally, the DUSP phosphatases are critically important regulators of MAPK signaling. They are also transcriptionally upregulated by mutant BRAF and KRAS and potentially highly relevant here (Cagnol, S. & Rivard, N. Oncogene 32:564-76 (2013); Huang, C. Y. & Tan, T. H. Cell Biosci 2:24 (2012); Low, H. B. & Zhang, Y. Immune Netw 16:85-98 (2016); Shen, J., et al. Cancer Med 5:2061-8 (2016)). Experiments are conducted to test the expression of the SPRY, SPRED, DUSP family members by further analysis of the RNA sequencing data obtained above and validated by Western as appropriate.

### Example 3: Characterize the Inflammatory Microenvironment and Contribution of Tumor Heterogeneity to PEASA

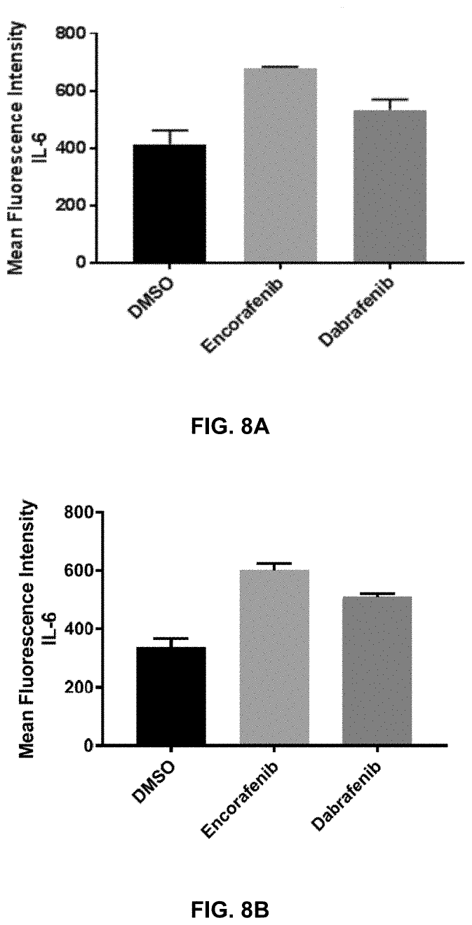

Because this mode of generating tumor cell senescence-like arrest is new, the nature of the secretory phenotype is unknown (Lasry, A. & Ben-Neriah, Y. Trends Immunol 36:217-28 (2015); Sieben, C. J., et al. Trends Cell Biol 28:723-737 (2018)). Oncogene-induced senescence shares features of therapy-induced senescence (IL-1, IL-6, CCL2) (Sieben, C. J., et al. Trends Cell Biol 28:723-737 (2018)) and classical senescence is associated with a secretory phenotype (SASP) (Rao, S. G. & Jackson, J. G. Trends Cancer 2:676-687 (2016)) and an inflammatory phenotype (SIR) (Pribluda, A., et al. Cancer Cell 24:242-56 (2013)). In characterizing the inflammatory phenotype conferred by BRAFi both IL-6 and PD-L1 were upregulated (FIGS. 7, 8). The presence of IL-6 is consistent with SASP, and PD-L1 suggests potential sensitivity to anti-PD1 axis immunotherapy. In this Example, experiments are conducted to characterize the secretome and inflammatory landscape of BRAFi-treated NRAS-mutant melanomas in cells initially and then in-vivo.

ELISA on PMA/ionomycin-activated cells and/or FACS is used to measure IL-1, IL-6, CCL2, MMP1/3, CXCL9/10, TLR1/2 as a mix of overlapping and distinct components of the SASP and the SIR (Lasry, A. & Ben-Neriah, Y. Trends Immunol 36:217-28 (2015)). Results are also validated with PD-L1/2 and CTLA-4 is measured. Immunosuppressive galectins 1, 3, 9, PGE2, and L-Arginase activity are quantified, as reported (Scarlett, U. K., et al. J Exp Med 209:495-506 (2012); Rutkowski, M. R., et al. Cancer Cell 27:27-40 (2015)).

It was recently shown that PTBP1 specifically regulates the immunosuppressive component of SASP (Georgilis, A., et al. Cancer Cell 34:85-102 e9 (2018); Schmitt, C.A. Cancer Cell 34:6-8 (2018)). Although it has not been shown that PEASA drives SASP, it is reasonable to posit some overlap, and experiments are conducted to probe for a PTBP1-regulated secretome. This is tested to directly to assess whether the key components of the PTBP1-regulated secretome (including IL-6, IL-la, IL-8) are specifically regulated as in the SASP, potentially identifying a means of augmenting anti-tumor immunity and avoiding the deleterious effects of immunosuppression.

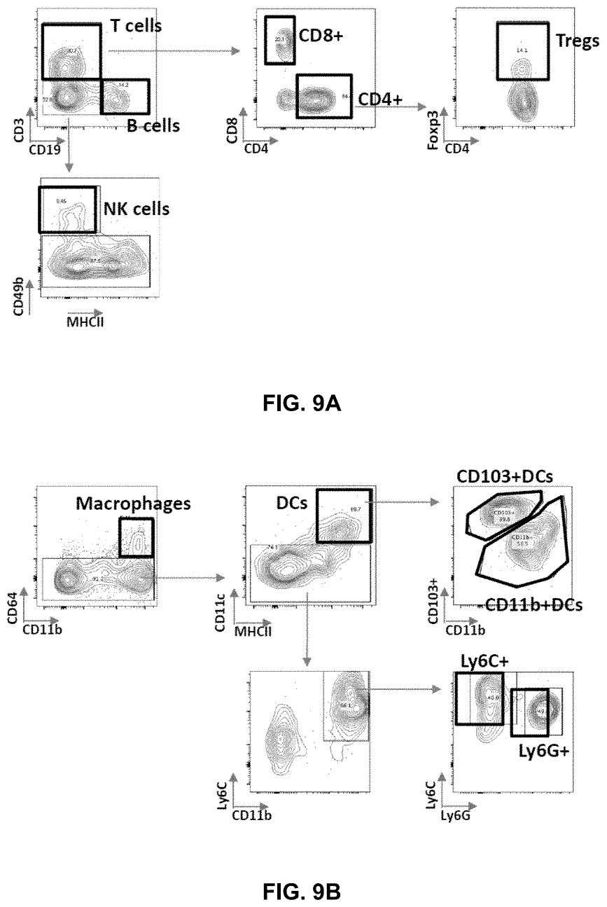

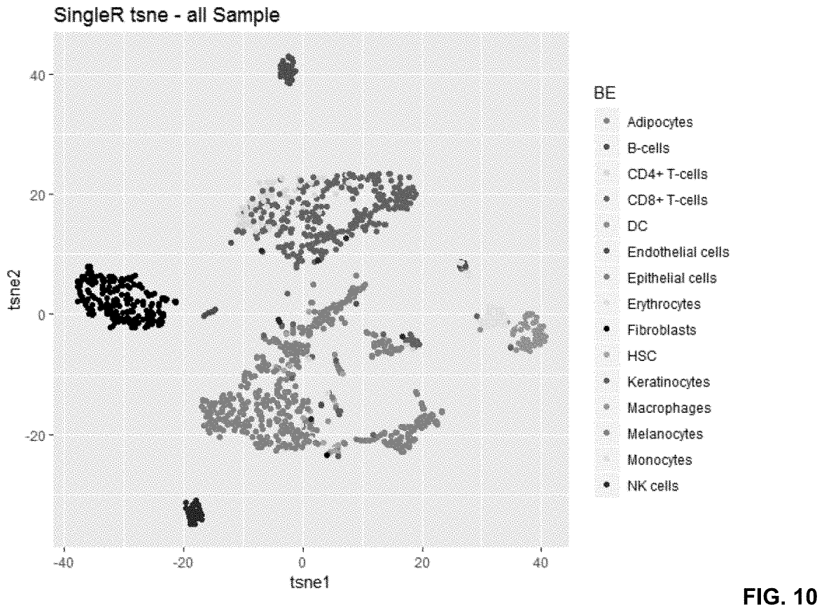

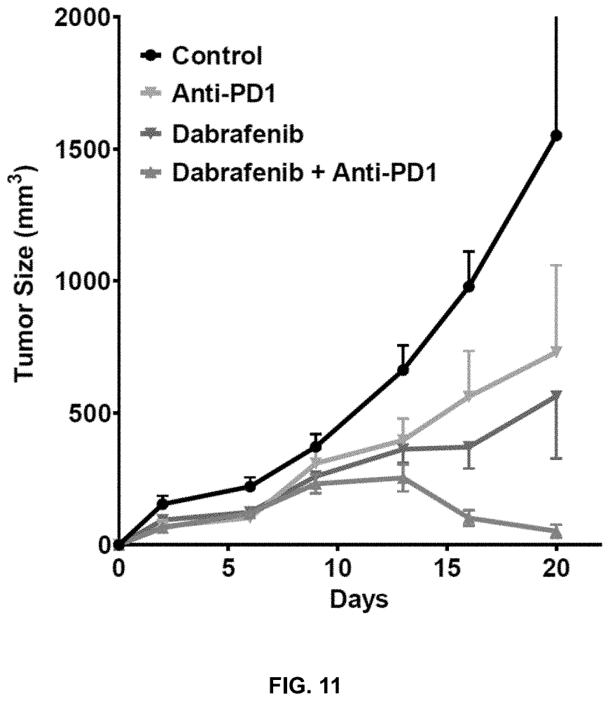

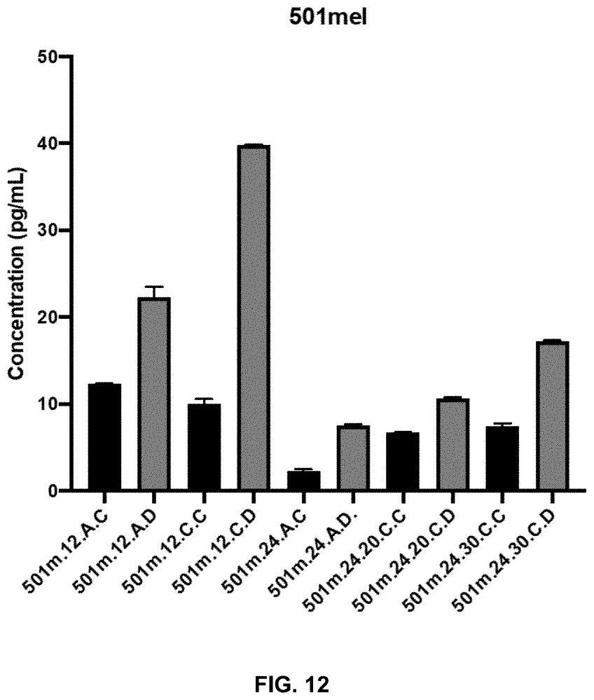

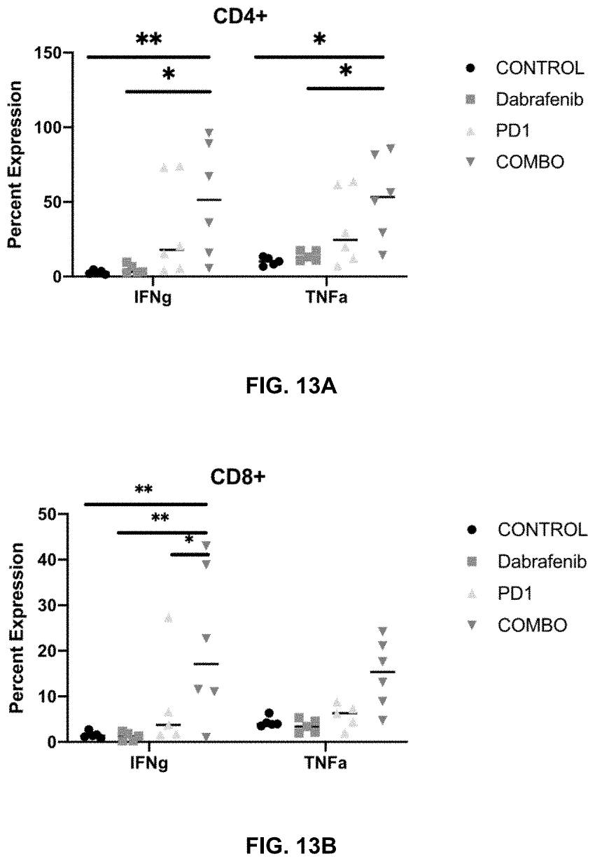

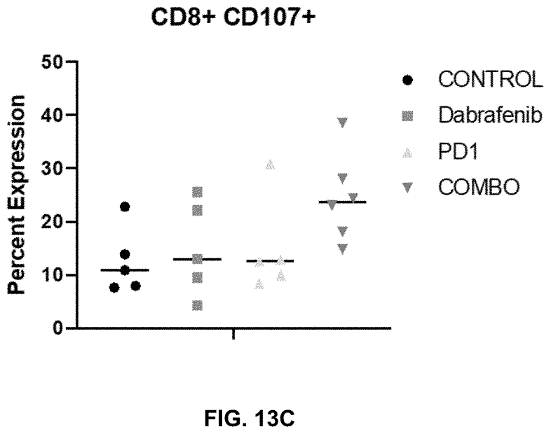

Next, experiments are conducted to identify how secreted components affect tumor infiltrating immune cells as BRAFi can increase antigen presentation (Boni, A., et al. Cancer Res 70:5213-9 (2010); Tompers Frederick, D., et al. Clin Cancer Res (2013)), T-cell activation through paradoxical ERK activation (Callahan, M. K., et al. Cancer Immunol Res 2:70-9 (2014)), T-cell infiltration, and IL-1β secretion (Khalili, J. S., et al. Clin Cancer Res 18:5329-40 (2012); Hajek, E., et al. Oncotarget 9:28294-28308 (2018)). Two C57BL/6 NRAS^{Q61R}-mutant melanoma lines are used (Burd, C. E., et al. Cancer Discov 4:1418-29 (2014))): 1-5 million cells are implanted within each flank. Once engrafted and grown to a size of 4 mm in largest dimension, mice are randomized into one of two arms: vehicle-treated and dabrafenib only (3.5 mg/kg/day); n=6 mice for each condition for a total of 24 mice), which are enrolled for 7 days of treatment with individual tumors tracked for size by caliper three times a week. At this point, the mice are harvested and tumors dissected, disaggregated and subjected to FACS analysis to identify specific subsets of cells (FIG. 9) and to identify differences between treated vs. control tumors. The specific cells profiled include lymphocytes, natural killer cells, dendritic cells including myeloid-derived dendritic cells, and macrophages. The use of n=6 mice in each condition is expected to have >98% power to distinguish changes of 2-fold at a significance of a=0.05.

Anti-tumor T cell immune responses are quantified using IFNγ and Granzyme B ELISPOT analysis of lymph nodes (Stephen, T., et al. Immunity 46:51-64 (2017); Zhu, H., et al. Cell Rep 16:2829-2837 (2016); Svoronos, N., et al. Cancer Discov 7:72-85 (2017); Perales-Puchalt, A., et al. Clin Cancer Res 23:441-453 (2017); Allegrezza, M. J., et al. Cancer Res 76:6253-6265 (2016)). A comprehensive analysis of activation (CD44, CD69, CD27, CD25) vs. exhaustion (PD-1, Lag3, T-bet/EOMES) markers in lymphocytes at tumor beds are included, along with the acquisition of a tissue-resident memory (CD103+CD69+) phenotype by tumor-infiltrating lymphocytes. Analysis is conducted to define to what degree the expected protective effects depend upon CD8 T cells by antibody-depleting them.

Nanostring can be used to probe the immune microenvironment around skin cancers (Feldmeyer, L., et al. Exp Dermatol 25:245-7 (2016)) using single cell RNAseq (FIG. 10). The functional changes accompanying BRAFi-treatment in both tumor cells and in the surrounding inflammatory infiltrate in-vivo are probed using unbiased single-cell RNA sequencing (10× Genomics, Moffitt Genomics Core) on BRAFi vs. control-treated NRAS^{Q61R}-mutant melanomas (Burd, C. E., et al. Cancer Discov 4:1418-29 (2014)) (n=2 lines×2 drug conditions each×3 replicates=12 samples). 1000-2000 cells sequenced to a depth of 50,000 to 100,000 reads per cell are interrogated using principal component analysis and t-Distributed Stochastic Neighbor Embedding (t-SNE) to segregate distinct cell populations (FIG. 10). As an exploratory approach, experiments begin with 3 replicates and expand to n=6 as above to achieve >98% power to distinguish changes of 2-fold at a significance of a=0.05.

Upon identification of the cell subsets most affected in terms of the largest gene expression differences which suggest changes in differentiation, activation status, and proliferation, an in-vitro system is used to test the effects of soluble factors to the phenotypes observed. Two key components of tumor immune infiltrates: T-cells and myeloid derived suppressor cells (MDSCs), can be involved. These two cell populations represent important components of the tumor infiltrating immune microenvironment and are logical targets for manipulation in enhancing cancer immunotherapy. conditioned media from both sensitive and insensitive lines are incubate with human CD4+ and CD8+ T-cells isolated from healthy human donors and their ability to proliferate and undergo activation upon CD3/CD28 stimulation is tested. IFNγ release is measured via FACS. The contribution of the conditioned media to affect in-vitro differentiation of human MDSCs from blood and excess normal bone marrow aspirates (Svoronos, N., et al. Cancer Discov 7:72-85 (2017); Allegrezza, M. J., et al. Cancer Res 76:6253-6265 (2016)) is also examined as well as their ability to suppress T-cell activation. Once it has been established how the secretome of BRAFi-treated NRAS-mutant melanoma cells affects T-cells and MDSCs (as a start), the candidate genes identified in RNA sequencing data will also be also validated in-vitro and in-vivo.

### Example 4: Identify New Combination Therapies with Immune Checkpoint Blockade

There is a tremendous need to generate effective and rigorous scientific rationales for combining targeted therapies and immunotherapies. Although dozens of trials are ongoing testing combination approaches with ICB, few are driven by mechanistically clear data. The disclosed data strongly implicate a potential growth arrest mechanism following exposure of RAS-mutant lines to BRAFi and potential synergy with anti-PD1 therapy (FIG. 11). In this Example, experiments are conducted to determine how the tumor immune environment may be modulated by PEASA to enhance responses to ICB in-vivo.

Once engrafted and grown to a size of 4 mm, mice are randomized into one of four arms (n=12 each; sham-treated, anti-PD1 antibody only (clone RMP1-14; 2 μg/μl thrice weekly), dabrafenib only (3.5 mg/kg/day), and the combination) for a total of 48 mice, which are enrolled for 30 days of treatment with individual tumors tracked for size. Toxicity is monitored by biweekly weight measurements and skin examination. The primary tumor endpoint is the sum of the largest dimensions of both lesions, with the estimated total volume used as sensitivity endpoints. With one-sided a=0.05, these numbers allow for detection of 2-fold decreases in the ratio of ratios (week 4 to baseline) in the sum of the largest linear dimensions of tumors in the combination (anti-PD1 and dabrafenib) versus placebo groups with powers of 97% or 83% if the coefficient of variation is 0.5 or 0.7, respectively. If statistically significant, similar tests are performed for the combination arm versus the single arm groups, using the Holm adjustment at a one-sided a of 0.05 to demonstrate superiority of the combination over individual therapies. In parallel, an identical trial using an anti-CTLA4 antibody is used. Following this initial characterization, experiments are conducted to test whether PEASA can sensitize tumors resistant to ICB by altering the sequencing of BRAFi before and after ICB.

Histologic and immunohistochemical assessment of cell cycle arrest (Ki67, p21), ERK activity (p-ERK), senescence (beta-galactosidase, H3K9me3) and apoptosis (cleaved caspase 3) in tumor cells as well as T-cell markers (CD4/8, GrzB, PD1, PD-L1/2, FOXP3, CTLA-4) is performed. Intensity scores using t-tests to be conducted at a=0.05. As above, single cell RNAseq is employed with similar parameters (FIG. 10). Gene expression differences are identified between individual immune subsets, confirmed with qRT-PCR and ultimately validated in-vivo validation using the transplantable model.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a RAS mutant cancer in a subject, comprising administering to the subject a therapeutically effective amount of a selective BRAF inhibitor (BRAFi) in combination with immunotherapy.

2. The method of claim 1 wherein the immunotherapy comprises a checkpoint inhibitor.

3. The method of claim 2, wherein the checkpoint inhibitor comprises an anti-PD-1 antibody, anti-PD-L1 antibody, anti-CTLA-4 antibody, or a combination thereof.

4. The method of claim 1, wherein the BRAFi comprises vemurafenib, dabrafenib, or encorafenib.

5. The method of claim 1, wherein the BRAFi and immunotherapy are administered simultaneously.

6. The method of claim 5, wherein the BRAFi and immunotherapy are in the same composition.

7. The method of claim 1, wherein the immunotherapy is administered at least 12 hours before the BRAFi.

8. The method of claim 1, wherein the BRAFi is administered at least 12 hours before the immunotherapy.

9. A composition comprising a selective BRAF inhibitor (BRAFi) and a checkpoint inhibitor in a pharmaceutically acceptable carrier.

10. The composition of claim 9, wherein the checkpoint inhibitor comprises an anti-PD-1 antibody, anti-PD-L1 antibody, anti-CTLA-4 antibody, or a combination thereof.

11. The composition of claim 9, wherein the BRAFi comprises vemurafenib, dabrafenib, or encorafenib.
